(19) United States
(12) Patent Application Publication
(10) Pub. No.: US 2011/0129844 A1
(43) Pub. Date: Jun. 2, 2011

(54) Use of Activating Transcription Factor-2 (ATF2) for Detecting Skin Cancer

(76) Inventors: Ze'ev Ronai, La Jolla, CA (US); Anindita Bhoumik, La Jolla, CA (US)

(21) Appl. No.: 12/810,008

(22) PCT Filed: Dec. 19, 2008
(86) PCT No.: PCT/US08/87816

§ 371 Date: Sep. 1, 2010

Related U.S. Application Data
(60) Provisional application No. 61/016,301, filed on Dec. 21, 2007.

(51) Int. Cl. C12Q 1/68

(52) U.S. Cl. 435/6.14

(57) ABSTRACT

The present invention is based on the discovery of the tumor suppressor role of activating transcription factor 2 (ATF2) in non-melanoma skin cancer development. Accordingly, the invention provides methods of diagnosing a subject as having or at risk of having skin cancer. Also provided are methods of treating skin cancer in a subject, characterizing skin cancer in a subject, and identifying agents useful for treating skin cancer in a subject.

### BACKGROUND OF THE INVENTION

1. Field of the Invention

The invention relates generally to skin cancer and more specifically to methods of detecting skin cancer in a subject using ATF2 activity or expression.

2. Background Information

Cancers are the second most prevalent cause of death in the United States, causing 450,000 deaths per year. One in three Americans will develop cancer, and one in five will die of cancer. While substantial progress has been made in identifying some of the likely environmental and hereditary causes of cancer, there is a need for substantial improvement in the diagnosis and therapy for cancer and related diseases and disorders.

Melanoma is a serious form of skin cancer in humans. It arises from the pigment cells (melanocytes), usually in the skin. Melanoma is currently increasing at the fastest rate of all cancers in the United States. Without including melanoma in situ, it is the seventh most common serious cancer in the United States. The growth and metastasis of melanoma as well as its notorious resistance to therapy present major obstacles to its treatment.

A number of so-called cancer genes, i.e., genes that have been implicated in the etiology of cancer, have been identified in connection with hereditary forms of cancer and in a large number of well-studied tumor cells. Cancer genes are broadly classified into “oncogenes” which, when activated, promote tumorigenesis, and “tumor suppressor genes” which, when damaged, fail to suppress tumorigenesis. While these classifications provide a useful method for conceptualizing tumorigenesis, it is also possible that a particular gene may play differing roles depending upon the particular allelic form of that gene, its regulatory elements, the genetic background and the tissue environment in which it is operating.

Typically, tumor suppressor genes are genes that, in their wild-type alleles, express proteins that suppress abnormal cellular proliferation. When the gene coding for a tumor suppressor protein is mutated or deleted, the resulting mutant protein or the complete lack of tumor suppressor protein expression may fail to correctly regulate cellular proliferation, and abnormal cellular proliferation may take place, particularly if there are coincidental perturbations of other cellular regulatory mechanisms. Thus, a need exists for the identification of a tumor suppressor gene associated with skin cancer in order to improve diagnosis and therapy.

### SUMMARY OF THE INVENTION

The present invention is based on the seminal discovery that the tumor suppressor activity of ATF2 is associated with skin cancer. Thus, ATF2 expression, activity and/or localization can be used to distinguish malignant skin cancer from non-malignant skin cancer.

Accordingly, the present invention provides methods of distinguishing melanoma from non-melanoma skin cancer in a subject. In one embodiment, the method includes distinguishing melanoma from non-melanoma skin cancer in a subject by comparing activating transcription factor 2 (ATF2) activity or expression in a first sample from the subject suspected of having skin cancer with ATF2 activity or expression in a normal tissue sample from the subject from a location distinct from the first sample, wherein a decrease in ATF2 activity or expression in the first sample as compared to the ATF2 activity or expression in the normal sample is diagnostic of melanoma in the subject.

In another embodiment, the method includes detecting subcellular localization of ATF2, wherein strong nuclear localization of ATF2 is diagnostic of melanoma in the subject. An increased level of ATF2 activity or expression in the test sample as compared to the ATF2 activity or expression in the normal sample is indicative of non-melanoma skin cancer in the subject. In another embodiment, the method includes detecting subcellular localization of ATF2, wherein strong cytosolic localization of ATF2 is diagnostic of non-melanoma skin cancer in the subject.

In another embodiment, the test sample is a skin sample. In another embodiment, the invention, further includes detecting increased expression of one or more genes selected from the group consisting of Plf2, Suclg1, Cav2, Syngr2, Mylc2b, Actn4, Eif4g2, Trappc6b, Napa, Elovl1, NUP35, H13, Cd44, Tm4sf8, Cdk4, Blnk, Atp6v0d1, Lamc2, Btg1, Gyk, Sumo1, Ablim1, 6030411K04Rik, Diap1, Dsg3, Fth1, Hnrpk, Psen1, Abcf2, Ppfia1, Prss11, Klk14, Ckap2, Fstl1, Tubb5, Dsc3, and Rhod, as compared to expression in the normal sample, thereby being indicative of melanoma in the subject. In another embodiment, the invention further includes detecting decreased expression of one or more genes selected from the group consisting of Itga6, PLA2, Gsdm1, Egln1, Pdrg1, Sprrl2, Csrp1, and Defb3, as compared to expression in the normal sample, thereby being indicative of melanoma in the subject. In another embodiment, the invention further includes detecting decreased expression of one or more of presenilin 1 (PS1) or Notch1, thereby being indicative of melanoma in the subject. In another embodiment, the invention further includes detecting increased expression of one or more of β-catenin, cyclin D1, c-Myc, epidermal growth factor receptor (EGFR), phospho-c-Jun (p-c-Jun) or JNK, thereby being indicative of melanoma in the subject.

In another aspect, the invention provides a method for diagnosing a subject has having or at risk of having melanoma. The method includes detecting subcellular localization of ATF2, wherein strong nuclear localization is indicative of melanoma. In another aspect, the invention provides a method for diagnosing a subject has having or at risk of having non-melanoma skin cancer. The method includes detecting subcellular localization of ATF2, wherein strong cytosolic localization is indicative of non-melanoma skin cancer.

In another embodiment, the test sample is a skin sample. In another embodiment, the invention, further includes detecting increased expression of one or more genes selected from the group consisting of Plf2, Suclg1, Cav2, Syngr2, Mylc2b, Actn4, Eif4g2, Trappc6b, Napa, Elovl1, NUP35, H13, Cd44, Tm4sf8, Cdk4, Blnk, Atp6v0d1, Lamc2, Btg1, Gyk, Sumo1, Ablim1, 6030411K04Rik, Diap1, Dsg3, Fth1, Hnrpk, Psen1, Abcf2, Ppfia1, Prss11, Klk14, Ckap2, Fstl1, Tubb5, Dsc3, and Rhod, as compared to expression in the normal sample, thereby being indicative of melanoma in the subject In another embodiment, the invention further includes detecting decreased expression of one or more genes selected from the group consisting of Itga6, PLA2, Gsdm1, Egln1, Pdrg1, Sprrl2, Csrp1, and Defb3, as compared to expression in the normal sample, thereby being indicative of melanoma in the subject. In another embodiment, the invention further includes detecting decreased expression of one or more of presenilin 1 (PS1) or Notch1, thereby being indicative of melanoma in the subject. In another embodiment, the invention further includes detecting increased expression of one or more of β-catenin, cyclin D1, c-Myc, epidermal growth factor receptor (EGFR), phospho-c-Jun (p-c-Jun) or JNK, thereby being indicative of melanoma in the subject.

In another embodiment, the invention provides a method for characterizing the stage or type of skin cancer in a subject. The method includes determining the ATF2 level or activity in a test sample from the subject suspected of having skin cancer and comparing the level to ATF2 levels or activities in samples from subjects of known skin cancer stage or type. When the ATF2 level or activity is about equal to the level or activity in the known skin cancer sample characterizes the skin cancer stage or type of the subject.

In another aspect, the invention provides a method for treating melanoma in a subject. The method includes administering to the subject an agent that reduces nuclear localization of ATF2. In one embodiment, ATF2 expression is shifted to cytosolic localization. In another embodiment, the method for treating skin cancer in a subject includes administering to the subject an agent that increases ATF2 activity or expression.

In another aspect, the invention provides a method for treating non-melanoma skin cancer in a subject. The method includes administering to the subject an agent that increases nuclear localization of ATF2. In one embodiment, ATF2 expression is shifted out of the cytosol and into the nucleus. In another embodiment, the method for treating skin cancer in a subject includes administering to the subject an agent that decreases ATF2 activity or expression.

In another aspect, the invention provides a method for monitoring a therapeutic regimen for treating skin cancer in a subject. In one embodiment, the invention includes determining a change in ATF2 level or activity during therapy. In another embodiment, the invention includes detecting a reduction in nuclear localization of ATF2.

In another aspect, the invention provides a method for identifying an agent that modulates ATF2 localization. The method includes contacting a test agent with a cell exhibiting strong nuclear expression of ATF2 and detecting a change in subcellular localization, as compared to the ATF2 localization prior to the contacting. A shift to cytosolic localization is indicative of an agent that modulates ATF2 localization, and identifies the agent of being useful for treating melanoma. A shift to nuclear localization is also indicative of an agent that modulates ATF2 localization, and identifies the agent of being useful for treating non-melanoma skin cancer. In one embodiment, the cell is a skin cancer cell, such as melanoma, squamous cell carcinoma, basal cell carcinoma, or spindle cell carcinoma. In another embodiment, the agent is a chemical compound.

In another aspect, the invention provides a method for identifying an agent that modulates ATF2 activity or expression. The method includes contacting a test agent with a cell expressing ATF2 and detecting a change in ATF2 activity or expression, as compared to the ATF2 activity or expression prior to the contacting. An increase in ATF2 activity or expression is indicative of an agent useful in treating melanoma. An decrease in ATF2 activity or expression is indicative of an agent useful in treating non-melanoma skin cancer. In one embodiment, the cell is a skin cancer cell, such as melanoma, squamous cell carcinoma, basal cell carcinoma, or spindle cell carcinoma. In another embodiment, the agent is a chemical compound.

### BRIEF DESCRIPTION OF THE DRAWINGS

FIGS. 1A to 1F are pictorial and graphical diagrams showing that targeted disruption of ATF2 in mouse skin increases susceptibility to papilloma formation in the two-stage chemical carcinogenesis.

FIG. 1A is a pictorial diagram showing the targeting strategy of wild-type allele of ATF2 encompassing exons 8 and 9 (boxes) and flanking loxP sequences (arrowheads).

FIG. 1B is a is a pictorial diagram showing expression of ATF2 by immunoblot in skin extracts from K14.ATF2^{wt/wt} (WT) and K14.ATF2^{f/f} mice. β-actin was used as load control. ATF2* indicates the fast migrating form of ATF2 released after deletion of exons 8 and 9. An ATF2 antibody which recognizes c-terminal epitopes was used for western blotting.

FIG. 1C is a pictorial diagram showing immunohistochemical analysis of the expression of ATF2 in the frozen skin sections of WT and K14.ATF2^{f/f} mice. Scale bar=50 μm. Arrows point to the expression of ATF2 in the epidermis of WT skin.

FIG. 1D is a pictorial diagram showing representative pictures of mice bearing papillomas 15 weeks after DMBA treatment.

FIG. 1E is a graphical diagram showing tumor incidence in the WT and K14.ATF2^{f/f} mice. Data represent the percentage of mice with skin papillomas; bars, SE. *statistically different from the WT mice (P<0.04) as determined by the student's t test.

FIG. 1F is a graphical showing average number of papillomas per mouse following DMBA/TPA treatment. Data represent an average number of skin papillomas per mouse; bars, SE. *statistically different from the WT mice (P<0.04) as determined by the student's t test.

FIGS. 2A to 2F are pictorial and graphical diagrams showing that epidermal hyperplasia is induced by TPA treatment.

FIG. 2A is a pictorial diagram showing dorsal skin of 8-week-old mice after three topical treatments with 10 μg of TPA was excised and stained with H&E. Histological analysis shown was performed 18 and 48 hrs after TPA treatment. Scale bar=50 μm. The upper panel shows the acetone treated control skin.

FIG. 2B is a graphical diagram showing the results from quantitative analysis of the epidermal thickness (μm) after TPA treatment. (P<0.001). Bars, SD (n=10). The thickness of the epidermis(μm) was calculated as described in the methods section.

FIG. 2C is a pictorial diagram showing immunohistochemical analysis of BrdU incorporation. The dorsal skin of 8-week-old mice received 3 topical treatments with 10 μg of TPA. 24 h post TPA treatment BrdU was injected i.p. and 1 h later the dorsal skin was isolated. Arrows indicate examples of BrdU-positive suprabasal cells. Scale bar=50 μm.

FIG. 2D is a graphical diagram showing the results from quantitative analysis of the BrdU positive cells after TPA treatment. (P<0.001). Bars, SD (n=10).

FIG. 2E is a graphical diagram showing the cell cycle profile of primary keratinocytes isolated from WT and K14.ATF2^{f/f} pups were subjected to FACS analysis (upper panel) and the percentages of cells in G₁, S, and G₂/M phases is shown in the lower panel (mean±SD of 3 experiments).

FIG. 2F is a pictorial diagram showing that apoptosis was assessed by using Active Caspase 3 antibody of skin from WT and K14.ATF2^{f/f} mice after 5 days of DMBA alone or DMBA and 18 h of TPA treatment. The TPA treatment was done for skin thickening to facilitate visualization of Active Caspase 3 positive cells in an Immunohistochemistry. Upper panel shows the western blotting with the Active caspase 3 antibody. The lower panel shows the staining pattern in the skin upon treatment with DMBA after 5 days followed by TPA treatment. Arrows indicate Active Caspase 3-positive cells.

FIGS. 3A to 3H are pictorial and graphical diagrams showing that a reduced level of presenillin-1 coincides with reduced Notch1 and elevated β-catenin expression in the TPA treated skin and papillomas of K14.ATF2^{f/f} mice.

FIG. 3A is a pictorial diagram showing epidermis of mice that received three topical applications of acetone or TPA (10 μg), was isolated 24 h after last treatment. Epidermis was isolated and proteins were prepared using RIPA buffer. Tissue lysates were subjected to Western blot with antibodies to presenilin-1, P-catenin, ATF2, c-Myc, cyclin D1 and EGFR. β-actin was used as loading control. Numbers reflect quantification of changes in PSI and β-catenin expression using LiCoR system.

FIG. 3B is a pictorial diagram showing the dorsal skin of 8-week-old mice subjected to treatment as indicated in panel a, was excised and paraffin sections were prepared and stained with β-catenin antibody. Bar=50 μm.

FIG. 3C is a pictorial diagram showing β-catenin staining in papillomas sections of the K14.ATF2^{f/f} and WT mice. Cells exhibiting nuclear β-catenin staining are marked with arrows. Scale bar=50 μm.

FIG. 3D is a pictorial diagram showing keratinocytes isolated from K14.ATF2^{f/f} and WT 1 d old pups were lysed and were subjected to immunoblot analysis with antibodies to Notch1. β-actin was used as loading control.

FIG. 3E is a pictorial diagram showing the dorsal skin of 8-week-old mice treated and prepared as indicated in panel b was immunostained with antibodies to Notch1. Bar=50 μm.

FIG. 3F is a pictorial diagram showing results from Western blots for phospho JNK and total JNK levels was performed as in FIG. 3A, except TPA was treated in all the cases. Two mice for each group is shown. β-actin was used as loading control.

FIG. 3G is a pictorial diagram showing immunohistochemical analysis of the expression of phospho c-Jun upon TPA treatment in the frozen skin sections of WT and K14.ATF2^{f/f} mice. Scale bar=50 μm. Arrows point to the expression of phospho c-Jun in the epidermis of WT skin.

FIG. 3H is a graphical diagram showing results from increased anchorage-independent cell growth in K14.ATF2^{f/f} keratinocytes. H-Ras^{V12} infected WT and K14.ATF2^{f/f} keratinocytes were seeded in soft agar. Colonies were counted 21 days later and scored microscopically. The data represent an average of three experiments. (P<0.005).

FIGS. 4A to 4D are pictorial and graphical diagrams showing reduced nuclear ATF2 expression in TMA samples from SCC and BCC patients.

FIGS. 4A and 4B are pictorial and graphical diagrams showing ATF2 protein expression levels were assessed using a tissue microarray that was stained with an antibody directed against ATF2. Examples of staining in normal skin, SCC, and BCC are shown (panel a, Scale bar=50 μm). Scoring of ATF2 nuclear and cytosolic localization was performed as detailed in Example 1. 40 samples from SCC patients, 14 samples of BCC patients and 10 normal matched and non-matched skin tissues were analyzed. Scores were divided into 4 categories: 1—scores ranging from 0-75; 2—scores ranging from 76-150; 3—scores ranging from 151-225 and 4—scores ranging from 226-300. The distribution of nuclear ATF2 staining in normal skin, BCC and SCC is shown in FIG. 4B.

FIG. 4C is a pictorial diagram showing staining of the skin derived from the WT mouse which developed papilloma with ATF2 (upper left) and β-catenin (lower left). Staining of the corresponding papillomas with antibodies to ATF2 (upper right) and β-catenin (lower right). Scale bar=50 μm.

FIG. 4D is a pictorial diagram showing staining of the TMA used in FIG. 4A with ATF2 (Left panel) and β-catenin antibody (Right panel). Scale bar=50 μm.

FIG. 5 is a pictorial diagram showing results from lysates from 3 f that were subjected to western blotting for Cyclin A and Cyclin B1. Two mice for each group are shown. β-actin was used as loading control.

### DETAILED DESCRIPTION OF THE INVENTION

The present invention is based on the discovery of the tumor suppressor, ATF2 and its association with skin cancer. ATF2 localization, expression and/or activity can be used to distinguish malignant skin cancer from non-malignant skin cancer.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

As used herein “corresponding normal cells” or “corresponding normal sample” refers to cells, or a sample from a subject, that is from the same organ and of the same type as the cells being examined. In one aspect, the corresponding normal cells comprise a sample of cells obtained from a healthy individual that does not have skin cancer or from a location on the skin of the same subject where the location is normal and does not contain a lesion and appears otherwise “normal” and disease free. Such corresponding normal cells can, but need not be, from an individual that is age-matched and/or of the same sex as the individual providing the cells being examined.

As used herein, the terms “sample” and “biological sample” refer to any sample suitable for the methods provided by the present invention. The sample of cells can be any sample, including, for example, a tumor sample obtained by biopsy of a subject having the tumor, a tumor sample obtained by surgery (e.g., a surgical procedure to remove and/or debulk the tumor), or a sample of the subject's bodily fluid. Thus, in one embodiment, the biological sample of the present invention is a tissue sample, e.g., a biopsy specimen such as samples from needle biopsy.

The term “skin” refers to the outer protective covering of the body, consisting of the epidermis (including the stratum corneum) and the underlying dermis, and is understood to include sweat and sebaceous glands, as well as hair follicle structures. Throughout the present application, the adjective “cutaneous” can be used, and should be understood to refer generally to attributes of the skin, as appropriate to the context in which they are used. In one embodiment, the skin is mammalian skin, such as mouse or human skin. The epidermis of the human skin comprises several distinct layers of skin tissue. The deepest layer is the stratum basalis layer, which consists of columnar cells. The overlying layer is the stratum spinosum, which is composed of polyhedral cells. Cells pushed up from the stratum spinosum are flattened and synthesize keratohyalin granules to form the stratum granulosum layer. As these cells move outward, they lose their nuclei, and the keratohyalin granules fuse and mingle with tonofibrils. This forms a clear layer called the stratum lucidum. The cells of the stratum lucidum are closely packed. As the cells move up from the stratum lucidum, they become compressed into many layers of opaque squamae. These cells are all flattened remnants of cells that have become completely filled with keratin and have lost all other internal structure, including nuclei. These squamae constitute the outer layer of the epidermis, the stratum corneum. At the bottom of the stratum corneum, the cells are closely compacted and adhere to each other strongly, but higher in the stratum they become loosely packed, and eventually flake away at the surface.

The terms “cell proliferative disorder” or “cellular proliferative disorder” refer to any disorder in which the proliferative capabilities of the affected cells is different from the normal proliferative capabilities of unaffected cells. An example of a cell proliferative disorder is neoplasia. Malignant cells (i.e., cancer) develop as a result of a multistep process. The term “malignant” refers to a tumor that is metastastic or no longer under normal cellular growth control.

The term “cancer” as used herein, includes any malignant tumor including, but not limited to, carcinoma and sarcoma. Cancer arises from the uncontrolled and/or abnormal division of cells that then invade and destroy the surrounding tissues. As used herein, “proliferating” and “proliferation” refer to cells undergoing mitosis. As used herein, “metastasis” refers to the distant spread of a malignant tumor from its sight of origin. Cancer cells may metastasize through the bloodstream, through the lymphatic system, across body cavities, or any combination thereof. The term “cancerous cell” as provided herein, includes a cell afflicted by any one of the cancerous conditions provided herein. The term “carcinoma” refers to a malignant new growth made up of epithelial cells tending to infiltrate surrounding tissues, and to give rise to metastases. The term “melanoma” refers to a malignant tumor of melanocytes which are found predominantly in skin but also in bowel and the eye. “Melanocytes” refer to cells located in the bottom layer, the basal lamina, of the skin's epidermis and in the middle layer of the eye. The term “non-melanoma,” when used in reference to skin cancer, refers to skin cancers that are slower growing and rarely metastesize. Exemplary non-melanoma skin cancers include, but are not limited to basal cell carcinoma (BCC) and squamous cell carcinoma (SCC).

A cell proliferative disorder as described herein may be a neoplasm. Such neoplasms are either benign or malignant, The term “neoplasm” refers to a new, abnormal growth of cells or a growth of abnormal cells that reproduce faster than normal. A neoplasm creates an unstructured mass (a tumor) which can be either benign or malignant. The term “benign” refers to a tumor that is noncancerous, e.g. its cells do not proliferate or invade surrounding tissues.

The term “pharmaceutically acceptable”, when used in reference to a carrier, is meant that the carrier, diluent or excipient must be compatible with the other ingredients of the formulation and not deleterious to the recipient thereof.

The terms “administration” or “administering” are defined to include an act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

Activating transcription factor 2 (ATF2) is a member of the bZIP family of transcription factors which is activated upon its phosphorylation by stress activated kinases in response to stress and cytokine stimuli (1-2). Transcriptional activity of ATF2 depends on its heterodimerization with members of the AP1 family, including c-Jun (3-5), or interaction with viral proteins, including v-Jim, E1A and the Tax proteins (6-8). ATF2 target genes include AP1 responsive genes, such as cyclin A, beta interferon and TNF alpha (9-11). Intriguingly, ATF2 has also been implicated in DNA damage response, through its phosphorylation by PIKK, including ATM (12). This phosphorylation is required for intra-S phase checkpoint control, and for its co-localization with components of the MRN complex within DNA damage repair foci.

As used herein, the term “protein” refers to at least two covalently attached amino acids, which includes proteins, polypeptides, oligopeptides and peptides. A protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration.

As used herein, the term “nucleic acid” means DNA, RNA, single-stranded, double-stranded or triple stranded and any chemical modifications thereof. Virtually any modification of the nucleic acid is contemplated. A “nucleic acid” can be of almost any length, from 10, 20, 30, 40, 50, 60, 75, 100, 125, 150, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100,000, 150,000, 200,000, 500,000, 1,000,000, 1,500,000, 2,000,000, 5,000,000 or even more bases in length, up to a full-length chromosomal DNA molecule. For methods that analyze expression of a gene, the nucleic acid isolated from a sample is typically RNA.

The role of ATF2 in stress and DNA damage response suggests that this protein could also play a role in tumorigenesis. Consistent with this possibility are earlier studies from which have suggested an important role of ATF2 in melanoma development and progression. Nuclear localization of ATF2 in tumor cells coincides with poor prognosis in melanoma patients (13). In addition, peptides derived from the N-terminal region of ATF2 efficiently repressed ATF2 function and reduced growth and metastasis of melanoma tumor cells in mouse models (14-17).

Accordingly, in one aspect, the invention provides methods of characterizing skin cancer as being either melanoma or non-melanoma. The data provided herein identifies opposing functions exhibited by ATF2 in melanoma or non-melanoma. As shown herein, ATF2 functions as a tumor suppressor in non-melanoma skin cancers, and is primarily located in cytosolic (i.e., non-nuclear fractions). In contrast, AFT2 functions as an oncogene and is primarily found in the nucleus of more aggressive melanomas, which coincides with poor prognosis. As used herein, the term “oncogene” refers to any gene that is a causative factor in the initiation of cancerous growth. As used herein, the term “tumor suppressor gene” refers to any gene whose activity stops the formation of tumors.

As such, in another aspect, the invention provides methods of treating melanoma by administering an agent that reduces nuclear localization of ATF2. For example, when administered to the subject, the agent will cause a shift in localization of ATF2 to the cytosol. The localization of proteins can be determined in a variety of ways by one of skill in the art. Generally, cells are examined for evidence of (1) a decrease in the amount of the protein in an origin cellular subregion; (2) an increase in the amount of the protein in a destination cellular subregion (or in an intermediate destination cellular subregion); and/or (3) a change in the distribution of the protein in the cellular subregions of the cell. The evidence can be direct or indirect. An example of indirect evidence is the detection of a cellular event mediated by the protein including, but not limited to, the cellular events discussed below.

As used herein, the term “ameliorating” or “treating” means that the clinical signs and/or the symptoms associated with the cancer or melanoma are lessened as a result of the actions performed. The signs or symptoms to be monitored will be characteristic of a particular cancer or melanoma and will be well known to the skilled clinician, as will the methods for monitoring the signs and conditions. For example, the skilled clinician will know that the size or rate of growth of a tumor can monitored using a diagnostic imaging method typically used for the particular tumor (e.g., using ultrasound or magnetic resonance image (MRI) to monitor a tumor).

In another aspect, the invention provides methods of treating non-melanoma skin cancer by administering an agent that increases nuclear localization of ATF2. For example, when administered to the subject, the agent will cause a shift in localization of ATF2 out of the cytosol and into the nucleus. An exemplary method for determining the localization of a protein of interest is by detection of a colorimetric change, for example, by visual observation. Various methods of visual observation can be used, such as light microscopy, fluorescence microscopy, and confocal microscopy. If desired, an epifluorescence microscope with a CCD camera can be used to measure translocation in the assays described below. This procedure can be automated, for example, by computer-based image recognition. The intracellular distribution of the protein can be determined by staining a cell with a stain specific for the protein. The stain comprises a specific binding substance, which binds specifically to the targeted protein. Examples of such a stain include, but are not limited to, antibodies that specifically bind to the protein. A stain specific for, e.g., ATF2, can be prepared using known immunocytochemistry techniques. In one embodiment, the stain further comprises a labeling moiety.

There are many different labels and methods of labeling known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds.

Those of ordinary skill in the art will know of other suitable labels for binding to the antibody, or will be able to ascertain such, using routine experimentation. The labeling moiety will be visibly observable in conventional immunohistochemical detection techniques being, for example, a fluorescent dye such as fluorescein, a chemiluminescense reagent, a radioisotope, a colloidal label, such as colloidal gold or colored latex beads, an enzyme label, or any other known labeling complex. Exemplary labels include, but are not limited to Cy3 and Cy5.

Suitable antibodies can be prepared using conventional antibody production techniques. The antibodies can be monoclonal or polyclonal. Antibody fragments, such as, for example Fab fragments, Fv fragments, and the like, are also contemplated. The antibodies can also be obtained from genetically engineered hosts or from conventional sources. Techniques for antibody production are well known to the person of ordinary skill in the art and examples of such techniques can be found in Harlow and Lane, Antibodies: A Laboratory Manual, Cold Spring Harbor Laboratory Press, Cold Spring Harbor (1988), Birch and Lennox, Monoclonal Antibodies: Principles and Applications, Wiley-Liss, New York (1995).

The role of the AP 1 transcriptional complex in skin carcinogenesis has been addressed previously. Specifically, c-Jun family members were shown to play an important role in skin cancer development, as K14-driven expression of the TAM67 dominant negative Jun family construct in the basal layer of the epidermis blocked TPA or UV-B induced tumors in a skin carcinogenesis model (18-20). In addition, mice in which c-Jun or JNK2 has been deleted exhibit marked reduction in skin cancer development (21-23).

Here, the role of ATF2 in a mouse skin carcinogenesis model was directly assessed by using a conditional KO of ATF2 in keratinocytes. Unlike the oncogenic role for c-Jun and JNK2, the present invention reveals that lack of ATF2 function contributes to accelerated development of papillomas, thereby suggesting a tumor suppressor role of ATF2 in keratinocytes.

Accordingly, in another aspect, the present invention provides methods of diagnosing a subject as having or at risk of having melanoma skin cancer. The method includes comparing ATF2 activity or expression in a test sample from the subject with ATF2 activity or expression in a normal sample preferably from a different location on the same subject. A decreased level of ATF2 activity or expression in the test sample as compared to the ATF2 activity or expression in the normal sample is indicative of melanoma in the subject. The invention also includes detecting increased or decreased expression of one or more genes shown in Table 1, as compared to expression in a normal sample. In another embodiment, the invention further includes detecting decreased expression of one or more of presenilin 1 (PS1) or Notch1. In another embodiment, the invention further includes detecting increased expression of one or more of β-catenin, cyclin D1, c-Myc, epidermal growth factor receptor (EGFR), phospho-c-Jun (p-c-Jun) or JNK.

In another aspect of the invention, a method for identifying an agent useful for treating skin cancer is provided. An agent useful in any of the methods of the invention can be any type of molecule, for example, a polynucleotide, a peptide, a peptidomimetic, peptoids such as vinylogous peptoids, a small organic molecule, chemical compound, or the like, and can act in any of various ways to treat skin cancer. For example, an agent useful for treating melanoma in a subject can increase expression or activity of ATF2, or cause ATF2 expression to be shifted to cytosolic localization. An exemplary agent useful for treating non-melanoma skin cancer in a subject will increase nuclear localization of ATF2 expression.

The agent can be administered in any way typical of an agent used to treat the particular type of cancer, or under conditions that facilitate contact of the agent with the target tumor cells and, if appropriate, entry into the cells. Entry of a polynucleotide agent into a cell, for example, can be facilitated by incorporating the polynucleotide into a viral vector that can infect the cells. If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A peptide agent can be introduced into a cell by various methods, including, for example, by engineering the peptide to contain a protein transduction domain such as the human immunodeficiency virus TAT protein transduction domain, which can facilitate translocation of the peptide into the cell. Generally, an agent is formulated in a composition (e.g., a pharmaceutical composition) suitable for administration to the subject. Such formulated agents are useful as medicaments for treating a subject suffering from melanoma or non-melanoma skin cancer. Thus, the agents identified will bear a tissue-specific effect depending on the type of cancer being treated.

Candidate agents encompass numerous chemical classes, though typically they are organic molecules, and often are small organic compounds (i.e., small molecules) having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In another aspect, the methods of the invention are useful for providing a means for practicing personalized medicine, wherein treatment is tailored to a subject based on the particular characteristics of the cancer in the subject. The method can be practiced, for example, by first determining whether the cancer is melanoma or non-melanoma skin cancer, as described above. An agent useful in treating a subject having melanoma is thereafter identified by contacting a sample of cells from the subject with at least one test agent, wherein a decrease in ATF2 activity or expression in the presence of the test agent as compared to the ATF2 activity or expression in the absence of the test agent identifies the agent as useful for treating the disease. Likewise, a detectable shift to cytosolic localization of ATF2 expression in the presence of the test agent as compared to localization of ATF2 expression in the absence of the test agent identifies the agent as useful for treating the disease. In contrast, an agent useful in treating a subject having non-melanoma skin cancer is identified by contacting a sample of cells from the subject with at least one test agent, wherein an increase in ATF2 activity or expression in the presence of the test agent as compared to the ATF2 activity or expression in the absence of the test agent identifies the agent as useful for treating the disease. Likewise, a detectable shift to nuclear localization of ATF2 expression in the presence of the test agent as compared to localization of ATF2 expression in the absence of the test agent identifies the agent as useful for treating the disease.

The sample of cells examined according to the present method can be obtained from the subject to be treated, or can be cells of an established cancer cell line of the same type as that of the subject. In one aspect, the established cell line can be one of a panel of such cell lines, wherein the panel can include different cell lines of the same type of disease and/or different cell lines of different diseases associated with nuclear localization of ATF2 expression. Such a panel of cell lines can be useful, for example, to practice the present method when only a small number of cells can be obtained from the subject to be treated, thus providing a surrogate sample of the subject's cells, and also can be useful to include as control samples in practicing the present methods.

Once disease is established and a treatment protocol is initiated, the methods of the invention may be repeated on a regular basis to monitor the expression level of ATF2, the activity of ATF2, and/or the subcellular localization of ATF2 in the subject. The results obtained from successive assays may be used to show the efficacy of treatment over a period ranging from several days to months. Accordingly, another aspect of the invention is directed to methods for monitoring a therapeutic regimen for treating a subject having skin cancer. A comparison of the expression level or activity of ATF2 prior to and during therapy will be indicative of the efficacy of the therapy. Therefore, one skilled in the art will be able to recognize and adjust the therapeutic approach as needed.

The efficacy of a therapeutic method of the invention over time can be identified by an absence of symptoms or clinical signs of the cell proliferative disorder in a subject at the time of onset of therapy. In subjects diagnosed as having the cell proliferative disorder, the efficacy of a method of the invention can be evaluated by measuring a lessening in the severity of the signs or symptoms in the subject or by the occurrence of a surrogate end-point for the disorder.

All methods of treating skin cancer may further include the step of bringing the agent into association with a pharmaceutically acceptable carrier, which constitutes one or more accessory ingredients. Pharmaceutically acceptable carriers useful for formulating an agent for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

The route of administration of a composition containing the agents of the invention will depend, in part, on the chemical structure of the molecule. Polypeptides and polynucleotides, for example, are not particularly useful when administered orally because they can be degraded in the digestive tract. However, methods for chemically modifying polynucleotides and polypeptides, for example, to render them less susceptible to degradation by endogenous nucleases or proteases, respectively, or more absorbable through the alimentary tract are well known (see, for example, Blondelle et al., Trends Anal. Chem. 14:83-92, 1995; Ecker and Crook, BioTechnology, 13:351-360, 1995). For example, a peptide agent can be prepared using D-amino acids, or can contain one or more domains based on peptidomimetics, which are organic molecules that mimic the structure of peptide domain; or based on a peptoid such as a vinylogous peptoid. Where the inhibitor is a small organic molecule such as a steroidal alkaloid, it can be administered in a form that releases the active agent at the desired position in the body, or by injection into a blood vessel such that the inhibitor circulates to the target cells.

The total amount of a compound or composition to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the agent that modulates ATF2 activity or expression to treat skin cancer in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

The methods of the invention can be performed by contacting samples of cells ex vivo, for example, in a culture medium or on a solid support. Alternatively, or in addition, the methods can be performed in vivo, for example, by transplanting a cancer cell sample into a test animal (e.g., a nude mouse), and administering the test agent or composition to the test animal. An advantage of the in vivo assay is that the effectiveness of a test agent can be evaluated in a living animal, thus more closely mimicking the clinical situation. Since in vivo assays generally are more expensive, they can be particularly useful as a secondary screen, following the identification of “lead” agents using an in vitro method.

Thus, the present study provides genetic evidence for a suppressor role of ATF2 in skin cancer of low malignant (i.e., non-melanoma) potential. Using the K14.ATF2^{f/f} mice, it was demonstrated that the number and incidence of papillomas increases in the absence of a transcriptionally active ATF2 in the basal layer of the epidermis. Consistent with these finding, keratinocytes prepared from K14.ATF2^{f/f} mice that were infected with mutant ras oncogene exhibited a marked increase in their ability to grow on soft agar compared with their WT counterpart, suggesting a transformed phenotype in vitro. Important support for the finding in the mouse model used here comes from the analysis of human skin tumors; unlike the strong nuclear expression of ATF2 in normal skin, SCC and BCC samples exhibit a significantly reduced nuclear staining. The latter is also consistent with reduced expression of ATF2 found in papillomas developed in the WT animals, supporting the notion that ATF2 need to be inactivated to support skin tumor development. While the present study did not assess whether lack of transcriptionally active ATF2 could contribute also to a papilloma to carcinoma transition, the data from SCC and BCC where ATF2 is largely inactivated due to either lower expression or reduced nuclear localization, support such a role. It is of interest to note that loss of ATF2 transcriptional activities per se, or in combination with either DMBA or TPA, is not sufficient to promote papilloma formation, suggesting that ATF2 is contributing to changes elicited by initiating and promoting events in keratinocytes. Among changes observed are reduced apoptosis of DMBA-treated skin, and increased proliferation of TPA-treated skin, as well as of primary keratinocytes. Collectively, these finding reveal that loss of ATF2 transcriptional activities is associated with -and contributes to- skin tumor formation.

In another aspect, the invention provides methods of characterizing skin cancer in a subject. The method includes determining the ATF2 level or activity in a sample from the subject and comparing the level to ATF2 levels or activities in samples from subjects of known skin cancer stages or cell types. An ATF2 level or activity equal to the level or activity of a corresponding known sample characterizes the skin cancer as being about equal to the known sample. Thus, differing levels of ATF2 activity or expression may be used to identify cancer as melanoma or non-melanoma skin cancer or even precancerous lesions, benign nevi and the like, and may further be used to determine and/or monitor progression of the stage or type of skin cancer in the subject.

The role of the API complex in the promotion of skin carcinogenesis has been demonstrated in mouse models in which the activity of c-Jun and other AP1 family members has been inhibited (18-20, 36-40). Similarly, JNK2 KO mice exhibit reduced skin tumor formation in the two-phase model (21). Surprisingly, unlike the promoting function of c-Jun and JNK2, ATF2 elicits suppressor function in the skin carcinogenesis process. The transcriptional activity of ATF2 is required for this tumor suppressor function, since the mouse model used in this study does not produce a transcriptionally active form of ATF2. The present invention therefore shows that PS1 and its targets β-catenin, EGFR, cyclin D1 and Notch1 are regulated by ATF2, and consequently suggests that their deregulation in the mutant mice is associated with the enhanced skin tumor phenotype seen.

Consistent with these findings is the observation that EGFR-JNK-c-Jun-Cyclin D1, which contributes directly to skin tumor progression are upregulated in the absence of transcriptionally active ATF2 (34,35,41,42). Furthermore, while the latter can be associated with loss of PS1 suppression, PS1 positively regulates Notch1, and reduced PS1 results in lower level of Notch1 expression, seen in the K14.ATF2^{f/f} mice tumors and tissues, as in skin tumor models, where it has been implicated as a tumor suppressor (43,44).

The gene expression microarrays which identified PS1 as an ATF2 target also identified other genes of interest including PHD2 (Egln1), HnRNPk and Sumo1. Changes in hypoxia-induced genes were recently reported to be associated with regulation by ATF2 (45) and would be consistent with the finding of altered PHD2 expression, which is a HIF1α regulatory protein (46).

When performed in a high throughput (or ultra-high throughput) format, the methods of the invention can be performed on a solid support (e.g., a microtiter plate, a silicon wafer, or a glass slide), wherein cell samples and/or genes of interest are positioned such that each is delineated from each other (e.g., in wells). Any number of samples (e.g., 96, 1024, 10,000, 100,000, or more) can be examined in parallel using such a method, depending on the particular support used. Where samples are positioned in an array (i.e., a defined pattern), each sample in the array can be defined by its position (e.g., using an x-y axis), thus providing an “address” for each sample. An advantage of using an addressable array format is that the method can be automated, in whole or in part, such that cell samples, reagents, genes of interest, and the like, can be dispensed to (or removed from) specified positions at desired times, and samples (or aliquots) can be monitored, for example, for ATF2 activity and/or the activities of any one or more of Plf2, Suclg1, Cav2, Syngr2, Mylc2b, Actn4, Eif4g2, Trappc6b, Napa, Elovl1, NUP35, H13, Cd44, Tm4sf8, Cdk4, Blnk, Atp6v0d1, Lamc2, Btg1, Gyk, Sumo1, Ablim1, 6030411K04Rik, Diap1, Dsg3, Fth1, Hnrpk, Psen1, Abcf2, Ppfia1, Prss11, Klk14, Ckap2, Fstl1, Tubb5, Dsc3, Rhod, Itga6, PLA2, Gsdm1, Egln1, Pdrg1, Sprr12, Csrp1, and Defb3, presenilin 1 (PS1), Notch1, β-catenin, cyclin D1, c-Myc, epidermal growth factor receptor (EGFR), phospho-c-Jun (p-c-Jun) and JNK.

Interestingly, expression of a dominant negative form of ATF2 in mouse skin spindle cell lines A5 and CarB resulted in inhibition of tumor formation using the immunodeficient SCID mouse model (47). The dominant negative ATF2 used in the above setting also impacted c-Jun transcriptional activities, which could have been responsible for the inhibition of xenograft growth in vivo (48). Furthermore, without limiting to certain theories, the difference between the AS and CarB cells used for the xenograft based analysis and the K14.ATF2^{f/f} mouse used herein may stem from the cell type in which ATF2 function was attenuated.

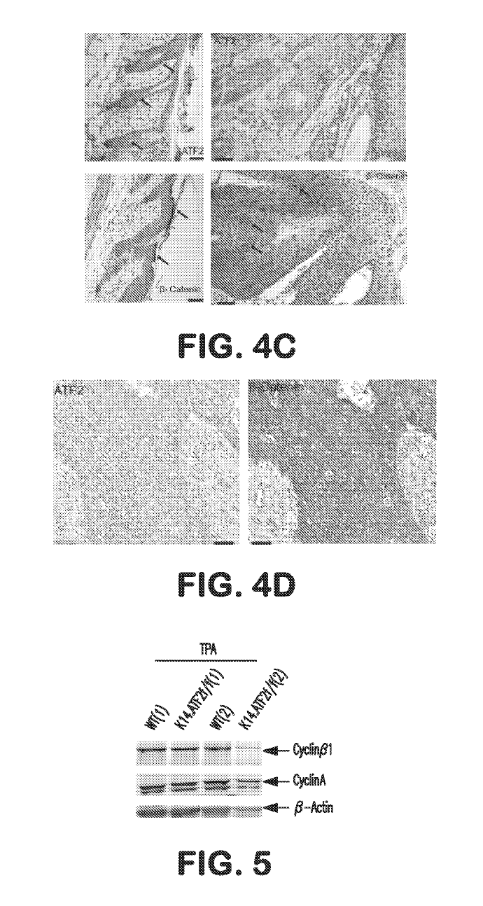

Consistent with these findings in the skin, ATF2 was recently implicated in eliciting a tumor suppressor function in mammary tumors (45). In contrast however, previous studies suggested a tumor promoting role of ATF2 in melanoma (12-17). Similarly, Notch1 functions as a tumor suppressor in mouse skin, as opposed to its function as an oncogene in other organs (44). Thus, tissue dependent expression of regulatory or accessory factors (i.e. heterodimeric transcription factors) is expected to alter the repertoire of genes that are regulated by ATF2 in keratinocytes versus other tissue types, including melanoma. Among possible mechanisms that could explain the different functions in the two tissue types is the altered expression and subcellular localization of ATF2 in skin tumors as opposed to melanoma. While in melanoma nuclear ATF2 expression is associated with poor prognosis (13), its nuclear localization is significantly reduced in BCC and SCC (FIG. 4). Clearly, different mechanisms regulate ATF2 expression and subcellular localization in the course of BCC and SCC, as opposed to melanoma development.

Overall, the present study provides the first genetic evidence for a suppressor role of ATF2 in skin cancer development, and suggest that PS1 is among target genes which may mediate this function. Decreased nuclear expression of ATF2 in human skin tumors further implicates loss of ATF2 function in keratinocyte transformation.

The materials of the invention are ideally suited for the preparation of a kit useful for the detection of ATF2. In one embodiment, the kit includes an antibody directed against ATF2, such as an ATF2 antibody which recognizes c-terminal epitopes. In one embodiment, the kit includes a carrier means being compartmentalized to receive in close confinement therein one or more containers such as vials, tubes, and the like, each of the container means comprising one of the separate elements to be used in the assay. For example, one of the container means may comprise a monoclonal antibody of the invention which is, or can be, detectably labelled. The kit may also have containers containing buffer(s) and/or a container comprising a reporter-means (for example, a biotin-binding protein, such as avidin or streptavidin) bound to a reporter molecule (for example, an enzymatic or fluorescent label).

The following examples are provided to further illustrate the advantages and features of the present invention, but are not intended to limit the scope of the invention. While they are typical of those that might be used, other procedures, methodologies, or techniques known to those skilled in the art may alternatively be used.

### EXAMPLE 1

The mouse knockout of ATF2 leads to early post-natal lethality (24). Thus, to study the function of ATF2 in the skin, the Cre-loxP system was utilized for disruption of the ATF2 gene in keratinocytes. Cre-dependent deletion of the ATF2 DNA binding domain and a portion of its leucine zipper, results in a transcriptionally inactive form of ATF2 (Breitwieser et al. unpublished results). Mice homozygous for the loxP-flanked (floxed) ATF2 gene (ATF2^{f/f}) were born at the expected Mendelian ratios and presented no obvious abnormalities. In addition, in a number of tissues that were analyzed, the levels of ATF2 expression were comparable between WT and ATF2^{f/f} (Data not shown).

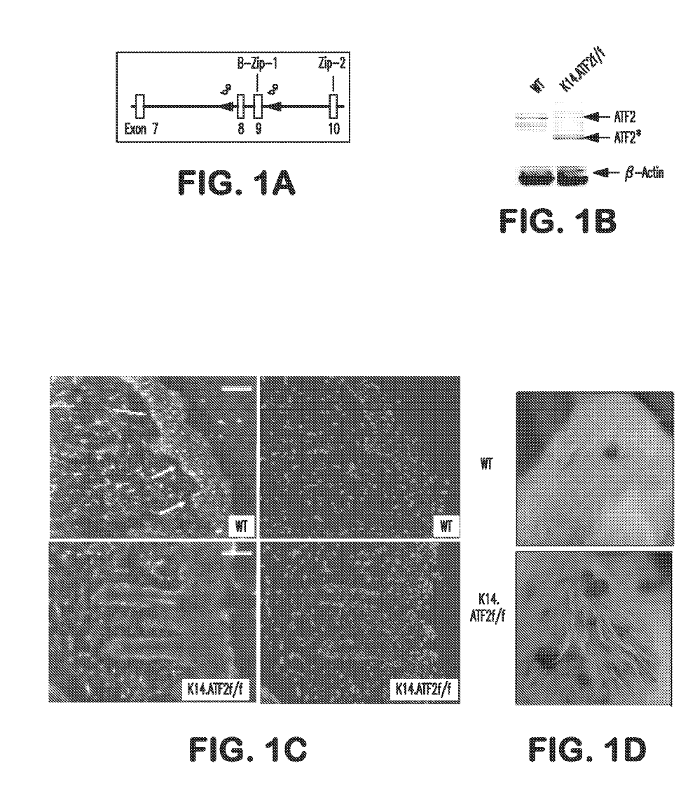

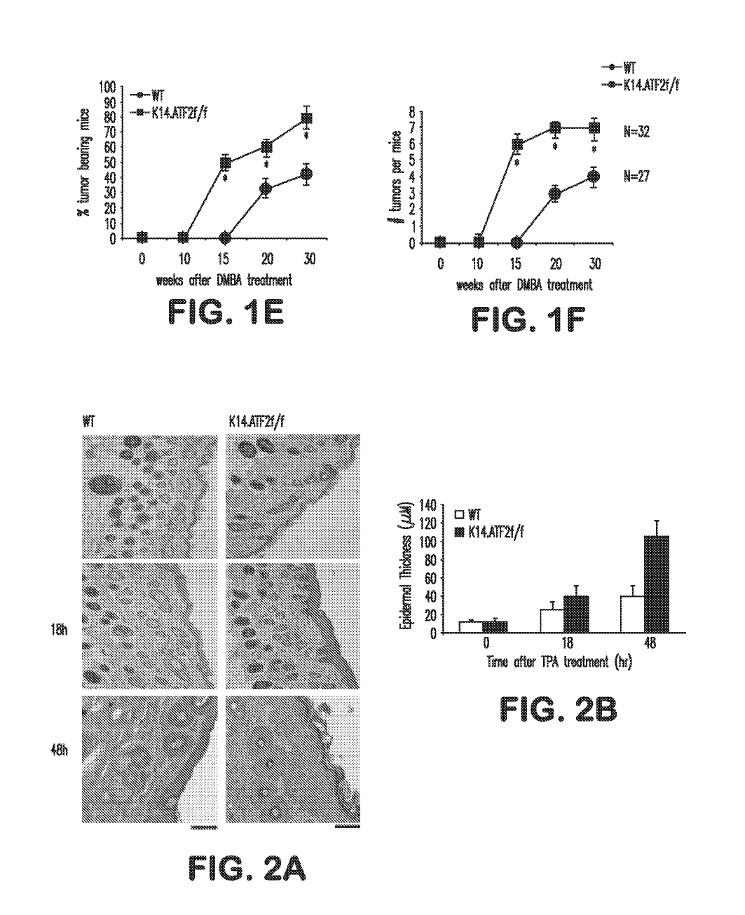

To elucidate the role of ATF2 in skin cancer, ATF2^{f/f} mice were crossed with keratin14-cre transgenic mice (K14-cre). The resulting ATF2^{f/f}/K14-cre (K14.ATF2^{f/f}) mice expressed the transcriptional mutant ATF2 gene in keratinocytes. Immunoblot analysis confirmed that keratinocytes prepared from wild-type express a 70 Kd band corresponding to full length ATF2 whereas keratinocytes of the K14.ATF2^{f/f} mice express a 55 Kd band, corresponding to ATF2 which lacks DNA binding and leucine zipper domains (FIG. 1B).

Animal treatment and tumor induction protocols. To study the function of ATF2 in the skin, the Cre-loxP system was utilized for disruption of the ATF2 gene in keratinocytes (40). The K14.ATF2^{f/f} mice and their littermate controls (WT) were of identical FVB/C57B1/6 genetic background. For tumor induction, mice were initiated with a dose of 10 μg of DMBA (Sigma) in 100 μl acetone applied to the dorsal surface 2 d after shaving. TPA (10 μg in 200 μl of acetone; Sigma) was applied every wk for 30 wk beginning 1 wk after initiation. The appearance of lesions in each mouse was monitored and recorded every week. Dorsal skin and/or Papillomas were dissected from euthanized mice and fixed in 10% neutral-buffered formalin for 48 hours and paraffin embedded. Sections (5 μm) were stained with H&E for histopathological analyses. Papilloma incidence and multiplicity were recorded weekly. Papilloma multiplicity was calculated as the average number of skin Papilloma per mouse. Papilloma incidence was calculated as the percentage of mice with skin Papilloma.

Immunohistochemistry. Skin specimens were fixed in neutral buffered formalin solution and processed for paraffin embedding. Skin sections (5 μm in thickness) were prepared and deparaffinized using xylene. For β-catenin, ATF2 and Notch1 immunostaining, tissue sections were incubated in DAKO antigen retrieval solution, for 20 min in a boiling bath, followed by treatment with 3% hydrogen peroxide for 20 min. Antibodies against ATF2 (1:100 from Santa Cruz), β-Catenin (1:500, Abeam), Notch-1 (1:100, Santa Cruz) were allowed to react with tissue sections at 4° C. overnight. Biotinylated anti-rabbit IgG was allowed to react for 30 min at room temperature and diaminobenzidine was used for the color reaction. Hematoxylin was used for counterstaining. The control sections were treated with normal mouse serum or normal rabbit serum instead of each antibody. For the Frozen sections the following antibodies were used-pc-Jun and ATF2 (1:100, Cell Signaling).

Immunohistochemical analysis revealed that ATF2 is expressed throughout the nucleated layers of epidermis and the dermis (FIG. 1C). Importantly, the use of the K14-cre transgene expression is limited to the basal layer of stratified squamous epithelia, thereby leaving ATF2 expression intact in neighboring tissues including the dermis (FIG. 1C).

Measurement of Hyperplasia. Hyperplasia was assessed in six WT and K14.ATF2^{f/f} mice (8-week-old) after treatment with acetone or TPA at the indicated time points. The thickness of the epidermis (μm) was measured using an image system (SlideBook-Intelligent imaging) in 15 fields per section.

Soft agar assay. Keratinocytes derived from WT and K14.ATF2^{f/f} newborn mice were infected with an H-ras^{V12} retrovirus. 16 h after infection 5×10³ cells were trypsinized to form a single-cell suspension and seeded in triplicate onto 6-well plates in MEM containing 0.35% agarose overlying a solidified 0.7% agarose. After the cell-containing layer solidified, 0.7% agarose was overlayed. Fresh growth medium was added every 4 days. Plates were incubated at 37° C. in 5% CO₂ for 21 days. Colonies (>0.5 mm) were counted under the microscope.

Statistical analysis. Data are shown as the means±SD. Unless indicated otherwise, statistical differences were determined using one-way ANOVA.

Preparation of epidermal cell lysates. The dorsal skin of the mice was excised and placed on a glass plate on ice, and the epidermis was removed with a razor blade and placed into RIPA lysis buffer. The lysis buffer contained 25 mM Tris-HCl pH 7.6, 150 mM NaCl, 1% NP-40, 1% sodium deoxycholate, 0.1% SDS, 1 mM phenylmethylsulfonyl fluoride, 1 μg/ml leupeptin, 1 μg/ml aprotinin, 1 mM Na₃VO₄ and 1 mM NaF. The lysates were incubated on ice for 10 min, snap frozen in liquid nitrogen, rethawed, and then centrifuged at 14,000 g for 15 min at 4° C.

5-bromo-2-deoxyuridine (BrdU) incorporation. 8-week-old K14.ATF2^{f/f} mice and WT mice were shaved 2 days before treatment with TPA (10 μg/200 μl acetone). BrdU (0.1 mg/g body weight) was injected intraperitoneally 1 h before the mice were sacrificed. The mice were euthanized 7, 24 and 48 h after six TPA treatments and their dorsal skins were removed, fixed in formalin and processed for paraffin embedding. Immunohistochemical staining was performed with a monoclonal anti-BrdU antibody (Sigma Aldrich, 1:100), which was applied to each section overnight at 4° C. after masking mouse IgG according to the protocol of the M.O.M. kit. Biotinylated anti-mouse IgG was used as the secondary antibody and the immunoreaction was visualized by the avidin-biotin-peroxidase complex immunostaining method using diaminobenzidine as the substrate. BrdU positive and negative basal cells were counted in three to five randomly selected areas of each skin section (3-4 sections per mouse) and the mean percentage of BrdU-positive cells and standard deviation (s.d.) for each treatment group were determined.

FACS analysis. Primary keratinocytes of WT and K14.ATF2^{f/f} mice were cultured for 3 days before cells were trypsinized, washed and fixed (70% ethanol in PBS). The cells were stained with propidium iodide (Sigma) and analyzed using a FACSCanto cell sorter using CELLQUEST software (Becton Dickinson). To determine percentages of cells in the G₁, S, and G₂/M phases original data were analyzed using ModFit LT software.

Microarray and real-time reverse transcription-PCR analyses. RNA from papillomas was isolated using the TRIZOL reagent (Sigma). Pooled RNA from the papillomas of two WT and three K14.ATF2^{f/f} papillomas were then hybridized in triplicate to the SENTRIX® Mouse-6 Expression BeadChip (Illumina) per manufacturer's recommendations. Data were quantile-normalized using the BeadExplorer Bioconductor package. A t-test was performed using the R (A language and environment for statistical computing statistics package) Development Core Team on the world wide web at R-project.org). To determine evidence for differential expression and p-values were corrected for multiple testing (48). To confirm differences in gene expression, real-time reverse transcription-PCR reactions were performed using Stratagene Mx3000p.

RNAs from WT and K14.ATF2^{f/f} papillomas were isolated using the TRIZOL reagent (Sigma). These RNAs were converted into Cy3- and Cy5-labeled cDNAs and hybridized with the SENTRIX® Mouse-6 Expression BeadChip (Illumina) according to manufacturer's recommendations. To confirm differences in gene expression, real-time reverse transcription-PCR reactions were performed using Stratagene Mx3000p.

Assessment of staining in skin tumors TMA. A skin cancer tissue array was purchased from US Biomax Inc (Rockville, Md.). The percentage of ATF2 positive tumor cells was determined semi-quantitatively by scoring the intensity of the immunostaining (49). The intensity was graded as: 0, negative; 1+, weak; 2+, moderate; 3+, strong. The immunoscore was determined by visual inspection (intensity X % of tumor stained=300 as a maximal score translated as 3 X intensity in 100% of the tissue). Scores were then divided into 4 categories: 1− scores ranging from 0-75, 2− scores ranging from 76-150, 3− scores ranging from 151-225 and 4− scores ranging from 226-300. Analysis was performed by 3 readers at Burnham Institute and Yale University. The slides were scanned with the slide scanner at 20× resolution (Aperio's ScanScope CS) and the images were retrieved using Imagescope viewing software.

Table 1: List of genes that were found to be upregulated or downregulated in gene profiling array performed on papillomas from ATF2 WT and K14.ATF2^{f/f} mice. Relative change in expression level is shown.

TABLE 1

| | Symbol | Accession No. | Fold Increase |
|---|---|---|---|
| Genes | Plf2 | NM_011118.1 | 4 |
| Upregulated | Suclg1 | NM_019879.1 | 5 |
| by ATF2 | Cav2 | NM_016900.2 | 2.5 |
| | Syngr2 | NM_009304.1 | 4 |
| | Mylc2b | NM_023402 | 3 |
| | Actn4 | NM_021895.2 | 4 |
| | Eif4g2 | NM_013507.2 | 5 |
| | Trappc6b | XM_127025.2 | 2.4 |
| | Napa | NM_025898.1 | 5 |
| | Elovl1 | NM_019422 | 5.4 |
| | NUP35 | AF411517.1 | 3 |
| | H13 | NM_010376.2 | 2.6 |
| | Cd44* | NM_009851.1 | 2.4 |
| | Cd44* | NM_009851.1 | 4 |
| | Tm4sf8 | NM_019793.2 | 5 |
| | Cdk4 | NM_009870.2 | 3.6 |
| | Blnk | NM_008528.3 | 3 |
| | Atp6v0d1 | NM_013477.2 | 4 |
| | Lamc2 | NM_008485 | 4 |
| | Btg1 | NM_007569.1 | 5.6 |
| | Gyk | NM_008194 | 2.6 |
| | Sumo1 | NM_009460.1 | 3.2 |
| | Ablim1 | NM_178688.2 | 2.6 |
| | 6030411K04Rik | BC018281 | 4 |
| | Diap1 | NM_007858.1 | 3.4 |
| | Dsg3 | NM_030596.1 | 2.8 |
| | Fth1 | NM_010239.1 | 4.8 |
| | Hnrpk | NM_025279.1 | 4.6 |
| | Psen1 | NM_008943.1 | 3 |
| | Abcf2 | NM_013853.1 | 4 |
| | Ppfia1 | XM_133979.4 | 2 |
| | Prssl1 | NM_019564.1 | 2.6 |
| | Klk14 | NM_174866.1 | 4 |
| | Ckap2 | XM_134100.3 | 2 |
| | Fstl1 | NM_008047.2 | 4 |
| | Tubb5 | NM_011655.2 | 6 |
| | Dsc3 | NM_007882.2 | 2 |
| | Rhod | NM_007485.2 | 3.2 |
| Genes | Itga6 | AK018033 | |
| downregulated | PLA2 | AK033572 | −2.6 |
| by ATF2 | Gsdm1 | NM_021347.2 | −2.4 |
| | Egln1 | NM_053207.1 | −3 |
| | Pdrg1 | NM_178939.1 | −2.5 |
| | Sprrl2 | NM_028625.1 | −2.6 |
| | Csrp1 | NM_007791.2 | −2.2 |
| | Defb3 | NM_013756.1 | −3.4 |

Bold- indicates the validated genes by Q-RTPCR or by Immunoblot.
*Denotes that the gene was detected twice in the profiling array. The differences between the hits are considered with the same range/significance.

Disruption of ATF2 increases susceptibility to skin carcinogenesis. To address the role of ATF2 in de novo skin carcinogenesis, the two-stage skin carcinogenesis protocol was used (25). In this model, tumors are initiated in epidermal keratinocytes by single topical application of the chemical carcinogen 7,12-dimethylbenz[a]anthracene (DMBA) with subsequent addition of the tumor promoter TPA over a period of 8-12 weeks. This procedure results in the development of benign papillomas with a high incidence of H-Ras mutations (25,26). Some of these tumors progress to squamous cell carcinomas, which can undergo epithelial-mesenchymal transition to spindle cell carcinomas. Significantly, K14.ATF2^{f/f} mice exhibited increased susceptibility to skin tumorigenesis with markedly accelerated kinetics of papilloma development (FIG. 1D). In K14.ATF2^{wt/wt} mice (WT), papillomas started to appear around 17 weeks after DMBA treatment (FIG. 1E) and by 30 weeks, about 40% had developed tumors. This slow development of papilloma development in littermate controls is attributed to their genetic background, FVB/C57B1/6 since mice of C57B1/6 background are more resistant to chemical-induced skin cancer (27). In contrast skin papillomas in K14.ATF2^{f/f} mice were observed as early as 11 weeks after DMBA treatment, with 50% of mice having developed tumors between weeks 13 and 15. Thus, compared with WT mice, the median appearance of skin papillomas is 5-6-weeks earlier in the ATF2 mutant keratinocytes [average±SE: 11.42±0.36 weeks (K14.ATF2^{f/f}; n=32) versus 17.63±0.30 weeks (WT; n=27); P<0.002] (FIG. 1E).

Importantly, the number of papillomas also increased in the K14.ATF2^{f/f} mice. By 15 weeks, K14.ATF2^{f/f} mice developed an average number of 6 tumors per mouse whereas the WT mice had none (FIG. 1F). These findings strongly suggest that the absence of functional ATF2 in keratinocytes confers increased sensitivity to skin cancer development and imply that ATF2 may elicit tumor suppressor function in the skin. Of note, treatment of DMBA or TPA alone did not result in papilloma development in WT or ATF2 mutant mice. This finding implies that lack of transcriptionally active ATF2 is not sufficient to augment initiation or promotion phases per se, but is important for the accelerated development of initiated lesions.

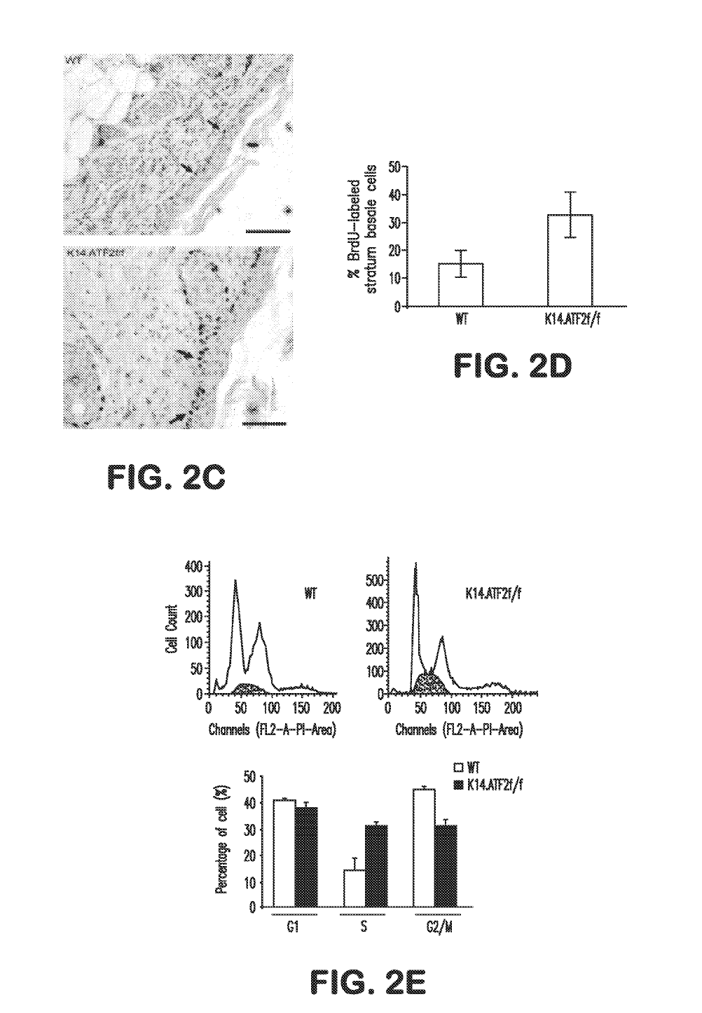

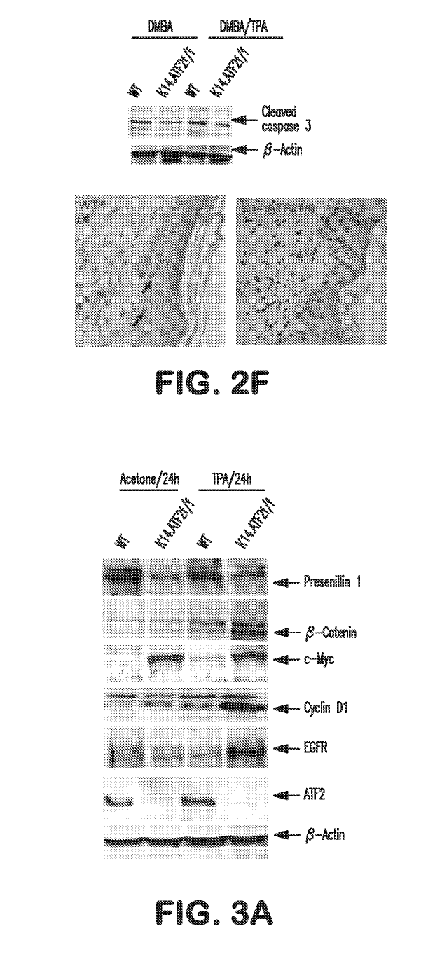

ATF2 deficiency increases epidermal hyperproliferation following addition of TPA. In order to determine the possible effect of ATF2 disruption on keratinocyte proliferation possible changes in epidermal hyperplasia was assessed. The number of nucleated cell layers in the untreated epidermis of WT and K14.ATF2^{f/f} mice was not significantly different (FIG. 2A, upper panel; FIG. 2B). Following TPA treatment, epidermal hyperplasia was induced in all genotypes, although the number of nucleated cell layers in the K14.ATF2^{f/f} mice was higher compared with the wild-type mice. Three topical applications of TPA (at 10 μg each) induced the formation of a hyperplastic epidermis consisting of a cell layer of 21±2.6 μm (mean±SD) thickness 18 hrs post treatment in WT mice compared with 42±3.2 μm in the K14.ATF2^{f/f} mice (FIG. 2A, middle panel; FIG. 2B). The difference was even more striking after 48 hrs, where the thickness of epidermal layers was 45±2.3 μm in wild-type mice versus 102±2.8 μm in K14.ATF2^{f/f} mice (FIG. 2A, Lower panel; FIG. 2B). These data reveal that the lack of transcriptionally active ATF2 in keratinocytes potentiates epidermal hyperplasia following exposure to a tumor promoter.

Induced DNA synthesis in basal epidermal cells of TPA-treated K14.ATF2^{f/f} mice. In light of the enhanced hyperplastic responses seen in the epidermis of K14.ATF2^{f/f} mice, possible changes in the rate of keratinocyte proliferation, which could account for the increased hyperplasia, were assessed. BrdU labeling was carried out to quantify DNA synthesis in basal keratinocytes. In wild-type mice, the percentage of BrdU-labeled basal cells was 5% prior to- and 15±3% 24 hours after- TPA treatment (FIG. 2C). Whereas K14.ATF2^{f/f} mice exhibited BrdU labeling that was similar to the WT animals (5%) prior to TPA treatment, 24 h after TPA treatment the K14.ATF2^{f/f} mice showed a significant increase in the number of BrdU-labeled cells [(32±3.0%), compared with WT ATF2 mice (15±3%) P<0.0045; (FIG. 2D)]. Further, primary cultures of K14.ATF2^{f/f} keratinocytes exhibit a marked increase in the S phase of the cell cycle, compared with the primary WT keratinocytes (FIG. 2E). These data suggest that the lack of transcriptionally active ATF2 causes increased cell proliferation in primary keratinocytes, and their hyperproliferation in the epidermis in response to treatment with tumor promoter.

Reduced apoptosis in epidermal keratinocytes of K14.ATF2^{f/f} mice. It was next determined whether higher incidence of papilloma formation could be attributed to altered rate of apoptosis, following DNA damage such as DMBA treatment. Reduced levels of active caspase 3 were observed in DMBA-treated K14.ATF2^{f/f} skin, compared with the skin of DMBA-treated WT mice, suggesting increased keratinocyte survival in K14.ATF2^{f/f} skin. This finding implies that absence of transcriptionally active ATF2 support a mutator phenotype, with consequent increase in tumorigenicity.

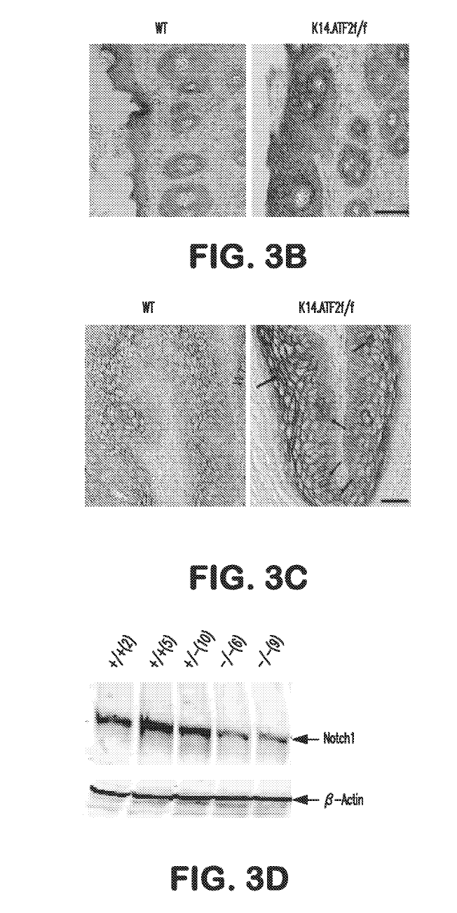

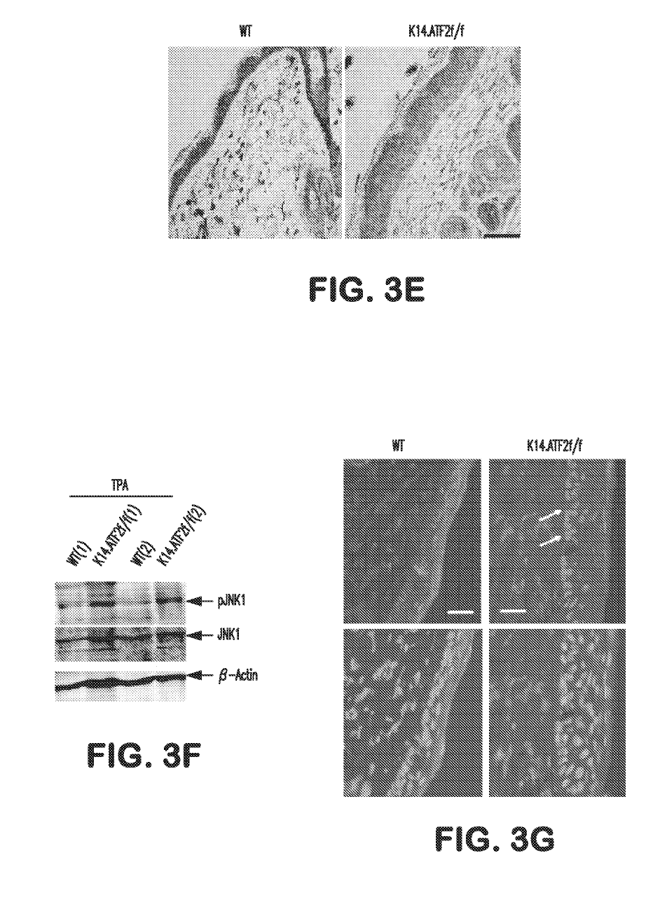

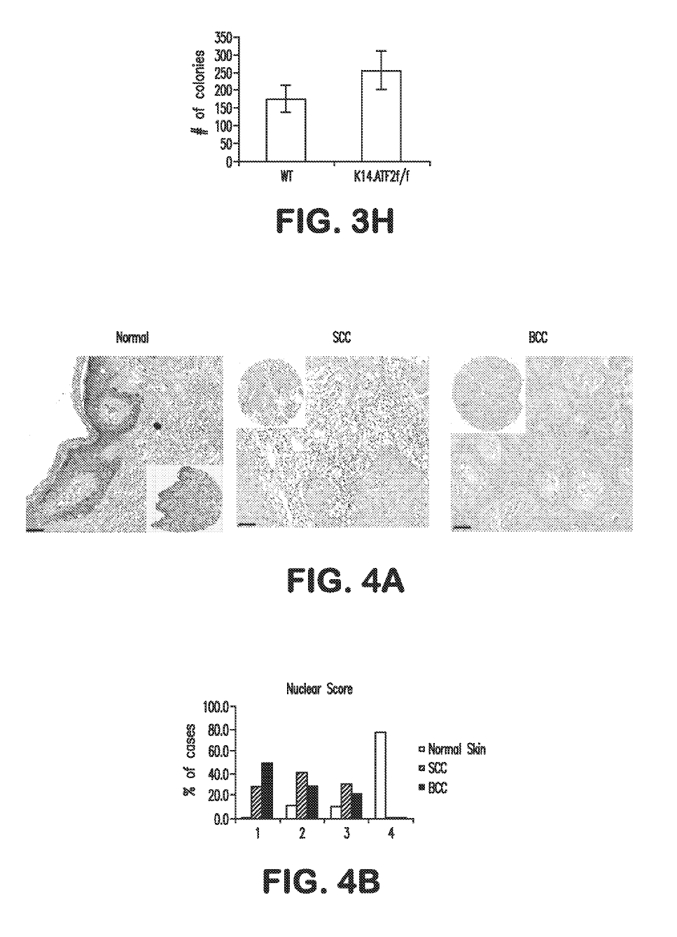

Reduced levels of presenilin 1 coincide with elevated β-catenin expression in the K14.ATF2^{f/f} papillomas. To elucidate the molecular pathways that were modified in the absence of transcriptionally potent ATF2, gene expression microarrays were employed. A total of 113 probes showed significant differential expression between WT and K14.ATF2^{f/f} mice (false discovery rate, 10%; Table 1; raw data available at NCBI GEO as accession GSE9328). Genes that were down-regulated in the K14.ATF2^{f/f} papillomas included presenilin 1 (PS1), which is required for the proteolytic processing of Notch during development and Alzheimer's disease pathogenesis (28). PS1 also serves as a scaffold protein which affects β-catenin phosphorylation and stability independently of the Wnt-regulated axin/CK1α complex (29). Interestingly PS1 knockout mice that are rescued through neuronal expression of the human PS1 transgene develop spontaneous skin cancers (30). PS1-null keratinocytes have higher cytosolic β-catenin and β-catenin/lymphoid enhancer factor-1/T-cell factor (β-catenin/LEF)-mediated signaling Consistent with these reports, epidermis of K14.ATF2^{f/f} mice not only exhibited reduced levels of presenilin 1, but also elevated level of β-catenin expression (FIG. 3A).

Consistent with the immunoblot analysis (FIG. 3A), β-catenin levels were found to be up-regulated in the epidermis of the K14.ATF2^{f/f} mice (FIG. 3B) and in papilloma samples (FIG. 3C). β-catenin staining was mostly localized to the membrane of the upper layer of the epidermis in both WT and K14.ATF2^{f/f} papillomas (FIG. 3C). Increased accumulation of cytosolic and nuclear β-catenin was observed in the basal layer of the epidermis of papillomas derived from the K14.ATF2^{f/f} mice. Since cell proliferation takes place within the basal cell layer of the epidermis, increased accumulation of cytosolic and nuclear β-catenin in the epidermis of K14.ATF2^{f/f} mice points to its possible contribution to increased epidermal hyperplasia and papilloma formation.

These data suggest that in the absence of transcriptionally active ATF2 there is a decrease in the expression of PS1 with concomitant increase in the expression of β-catenin.

Elevated cyclin D1 and c-Myc, and reduced Notchl expression in K14.ATF2^{f/f} tissues. As cyclin D1 and c-Myc are direct target genes for β-catenin/LEF signaling (31,32) changes in their levels of expression were assessed. Western blot analysis reveled that cyclin D1 protein was indeed increased in the epidermis of K14.ATF2^{f/f} mice (FIG. 3A) following TPA treatment. Basal levels of c-Myc were also upregulated in the epidermis of the K14.ATF2^{f/f} mice with a modest increase following TPA treatment (FIG. 3A). The finding that cyclin D1 and c-Myc, two of the major transcriptional targets of β-catenin/LEF signaling, are upregulated in epidermis of K14.ATF2^{f/f} mice shows that the increased β-catenin protein is functional.

Since PS1 is also implicated in the activation of the Notchl-signaling pathway (33) changes in the expression of processed (cleaved) Notch1 were analyzed. Lower levels of processed Notch1 expression were found in protein lysates prepared from the epidermis (data not shown) and in keratinocytes derived from the epidermis of K14.ATF2^{f/f} mice, compared with the WT mice (FIG. 3D). Interestingly, nuclear localization of Notchl was detected throughout the WT epidermis, but only in the upper layer of the epidermis of the K14.ATF2^{f/f} mice (FIG. 3E). These results suggest that the epidermis of K14.ATF2^{f/f} mice exhibits lower levels of PS1 expression, which is associated with lower levels of processed Notch1 and higher β-catenin expression.

Elevated EGFR, pJNK and p-C-Jun expression in K14.ATF2^{f/f} keratinocytes. As PS1 was also implicated in the negative regulation of epidermal growth factor receptor (EGFR) signaling and turnover (34,35) changes in EGFR levels in the skin of WT and ATF2 mutant mice were assessed. Western blot analysis revealed elevated levels of EGFR in K14.ATF2^{f/f} skin treated with TPA, compared with the WT counterpart (FIG. 3A).

Increased EGFR expression is expected to result in activation of respective downstream signaling pathways, including the activation of JNK and c-Jun. Immunohistochemistry of TPA treated K14.ATF2^{f/f} skin samples revealed increased levels of phospho c-Jun expression in the basal layers of the epidermis (FIG. 3G), consistent with increased activity of its kinase, JNK (FIG. 3F). Of note, c-Jun was also implicated in positive regulation of EGFR (20). These data identify changes in JNK-Jun signaling pathways and their upstream regulator, EGF receptor.

Increased anchorage-independent growth of Ras-infected K14.ATF2^{f/f} keratinocytes. A hallmark of malignant transformation is anchorage-independent growth. A soft agar assay was used to measure anchorage-independent growth of keratinocytes derived from the skin of WT and K14.ATF2^{f/f} newborn mice which were infected in culture with a mutant Ras oncogenes. Modest, albeit significant, increase was observed in the number (but not size) of colonies formed in the H-Ras^{v12} infected K14.ATF2^{f/f} keratinocytes, compared with the WT counterparts (FIG. 3H; P<0.005). These findings suggest that lack of ATF2 transcriptional activity increases the tumorigenic potential of keratinocytes in vitro as well as in vivo.

Reduced level and altered subcellular localization of ATF2 in skin cancer tissue microarrays. To characterize the expression of ATF2 in human skin cancer, a tissue microarray containing normal skin histospots and specimens from 40 patients with squamous cell carcinoma (SCC) or basal cell carcinoma (BCC) was employed. Immunohistochemical staining for ATF2 in normal skin revealed strong nuclear and moderate cytoplasmic expression in the basal layer of the epidermis, (FIG. 4A). To assess the differences in levels of ATF2 expression, an immunoscore-based analysis was applied (see above for details), and differences were assessed by un-paired t-tests. Significantly, in contrast to the predominant nuclear localization of ATF2 in normal skin, samples from malignant tissues (SCC and BCC) exhibited marked reduction of nuclear ATF2 expression (P=0.0003), while the cytoplasmic ATF2 expression was not significantly different (P=0.13) (FIG. 4B, and data not shown).

Decreased nuclear and variable cytosolic staining was seen in the SCC subset of specimens when compared with normal skin (FIG. 4B; P=0.0005 and P=0.194, respectively). Similarly, analysis of BCC subset of samples identified decreased nuclear ATF2 expression when compared with normal skin (P<0.0001) and variable cytoplasmic ATF2 expression (FIG. 4B; P=0.116). These data indicate that ATF2 expression is either decreased or shifted from the nucleus to the cytosol in the majority of human SCC and BCC samples. As both changes would result in reduced ATF2 transcriptional activities, these data suggest that ATF2 function is attenuated in these skin tumors.

Consistent with the finding in BCC and SCC samples, level of ATF2 expression was markedly reduced in the papillomas developed in the WT mice compared with the normal appearing skin (FIG. 4C). Conversely, the expression of β-catenin increased in these papillomas, consistent with the finding of elevated β-catenin expression in the K14.ATF2^{f/f} papillomas. Significantly, and in agreement with the data provided herein, an increase in β-catenin staining intensity was seen in samples from SCC and BCC (FIG. 4D). These findings establish that ATF2 transcriptional activity is attenuated due to reduced or altered (cytosolic) localization in papillomas and human skin cancers, which coincides with elevated β-catenin expression.

### REFERENCES

1. van Dam, et al., (1995) EMKO J 14:1798-1811.

2. Gupta, et al., (1995) Science 267:389-393.

3. van Dam, et al., (2001) Oncogene 20:2453-2464.

4. Benbrook, et al., (1990) Oncogene 5:295-302.

5. Kerppola, et al., (1993) Mol Cell Biol 13:5479-5489.

6. Huguier, et al., (1998) Mol Cell Biol 18:7020-7029.

7. Kawasaki, et al., (1998) Genes Dev 12:233-245.

8. Franklin, et al., (1993) J Biol Chem 268:21225-21231.

9. Nakamura, et al., (1995) Exp Cell Res 216:422-430.

10. Tsai, et al., (1996) Mol Cell Biol 16:5232-5244.

11. Falvo, et al., (2000) Mol Cell Biol 20:4814-4825.

12. Bhoumik, et al., (2005) Mol Cell 18:577-587.

13. Berger, et al., (2003) Cancer Res 63:8103-8107.

14. Bhoumik, et al., (2004) Proc Natl Acad Sci USA 101:4222-4227.

15. Bhoumik, et al., (2001) Clin Cancer Res 7:331-342.

16. Bhoumik, et al., (2002) J Clin Invest 110:643-650.

17. Bhoumik, et al., (2004) Cancer Res 64:8222-8230.

18. Young, et al., (1999) Proc Natl Acad Sci USA 96:9827-9832.

19. Li, et al., (1996) Cancer Res 56:483-489.

20. Zenz, et al., (2003) Dev Cell 4:879-889.

21. Chen, et al., (2001) Cancer Res 61:3908-3912.

22. Arnott, et al., (2002) Oncogene 21:4728-4738.

23. Matthews, et al., (2007) Cancer Res 67:2430-2438.

24. Maekawa, et al, (1999) J Biol Chem 274:17813-17819.

25. Yuspa SH (1994) Cancer Res 54:1178-1189.

26. Roop, et al., (1986) Nature 323:822-824.

27. DiGiovanni, et al., (1993) Carcinogenesis 14:319-321.

28. De Stooper, et al., (1999) Nature 398:518-522.

29. Kang, et al., (2002) Cell 110:751-762.

30. Xia, et al., (2001) Proc Natl Acad Sei USA 98:10863-10858.

31. Shtutman, et al., (1999) Proc Natl Acad Sci USA 96:5522-5527.

32. He, et al., (1998) Science 281:1509-1512.

33. Song, et al., (1999) Proc Natl Acad Sci USA 96:59-63.

34. Repetto, et al., (2007) Proc Natl Acad Sci USA. 104:10613-8.

36. Zoumpourlis, et al., (2000) Oncogene 19:4011-4021.

37. Bowden, et al., (1994) Cancer Res 54:1882s-1885s.

38. Dong, et al., (1994) Proc Natl Acad Sci USA 91:609-613.

39. Dong, et al., (1995) Carcinogenesis 16:749-756.

40. Finch, et al., (1996) Carcinogenesis 17:2551-2557.

41. Bianchi, et al., (1993) Oncogene 8:1127-1133.

42. Robles, et al., (1998) Genes Dev 12:469-2474.

43. Nicolas, et al., (2003) Nat Genet 33:416-421.

44. Lefort, et al., (2007) Genes Dev 21:562-577.

45. Maekawa, et al., (2007) Mol Cell Biol 27:1730-1744.

46. Berra, et al., (2003) EMBO J 22:4082-4090.

47. Papassava, et al., (2004) Cancer Res 64:8573-8584.

48. Gu, et al., (1993) Cell 73:1155-1164.

49. Benjamini, et al., (2001). Annals of Statistics 29:1165-1188.

50. Krajewska, et al., (2005) Clin Cancer Res 11:5451-5461.

Although the invention has been described with reference to the above example, it will be understood that modifications and variations are encompassed within the spirit and scope of the invention. Accordingly, the invention is limited only by the following claims.

1. A method of distinguishing melanoma from non-melanoma skin cancer in a subject, comprising comparing activating transcription factor 2 (ATF2) activity or expression in a first sample from the subject suspected of having skin cancer with ATF2 activity or expression in a normal tissue sample from the subject from a location distinct from the first sample, wherein a decrease in ATF2 activity or expression in the first sample as compared to the ATF2 activity or expression in the normal sample is diagnostic of melanoma in the subject.

2. The method of claim 1, wherein the test sample is a skin sample.

3. The method of claim 1, further comprising detecting increased expression of one or more genes selected from the group consisting of Plf2, Suclg1, Cav2, Syngr2, Mylc2b, Actn4, Eif4g2, Trappc6b, Napa, Elovl1, NUP35, H13, Cd44, Tm4sf8, Cdk4, Blnk, Atp6v0d1, Lamc2, Btg1, Gyk, Sumo1, Ablim1, 6030411K04Rik, Diap1, Dsg3, Fth1, Hnrpk, Psen1, Abcf2, Ppfia1, Prss11, Klk14, Ckap2, Fstl1, Tubb5, Dsc3, and Rhod, as compared to expression in the normal sample.

4. The method of claim 1, further comprising detecting decreased expression of one or more genes selected from the group consisting of Itga6, PLA2, Gsdm1, Egln1, Pdrg1, Sprr12, Csrp1, and Defb3, as compared to expression in the normal sample.

5. The method of claim 1, further comprising detecting decreased expression levels of presenilin 1 (PS1), decreased expression levels of Notch1, increased expression levels of β-catenin, increased expression levels of cyclin D1, increased expression levels of c-Myc, increased expression levels of epidermal growth factor receptor (EGFR), increased expression levels of phospho-c-Jun (p-c-Jun), increased expression levels of JNK, or any combination thereof, as compared to expression in the normal sample.

6. The method of claim 1, further comprising detecting decreased expression levels of presenilin 1 (PS1), as compared to expression in the normal sample.

7. The method of claim 1, further comprising detecting increased expression levels of β-catenin, as compared to expression in the normal sample.

8. The method of claim 1, further comprising detecting increased expression levels of cyclin D1, as compared to expression in the normal sample.

9. The method of claim 1, further comprising detecting increased expression levels of c-Myc, as compared to expression in the normal sample.

10. The method of claim 1, further comprising detecting decreased expression levels of Notch1, as compared to expression in the normal sample.

11. The method of claim 1, further comprising detecting increased expression levels of epidermal growth factor receptor (EGFR), as compared to expression in the normal sample.

12. The method of claim 1, further comprising detecting increased expression levels of phospho-c-Jun (p-c-Jun), as compared to expression in the normal sample.

13. The method of claim 1, further comprising detecting increased expression levels of JNK, as compared to expression in the normal sample.

14. The method of claim 1, further comprising detecting nuclear localization of ATF2 expression.

15. The method of claim 1, wherein an increase in ATF2 activity or expression in the test sample as compared to the ATF2 activity or expression in the normal sample is diagnostic of non-melanoma skin cancer in the subject.

16. The method of claim 15, further comprising detecting cytosolic localization of ATF2 expression.

17. The method of claim 15, wherein the skin cancer is squamous cell carcinoma, basal cell carcinoma, or spindle cell carcinoma.

18. A method of diagnosing a subject as having or at risk of having melanoma, comprising detecting subcellular localization of activating transcription factor 2 (ATF2) in a test sample from the subject, wherein nuclear localization of ATF2 is diagnostic of skin cancer in the subject.

19-20. (canceled)

21. A method for characterizing the stage or type of skin cancer in a subject comprising determining the ATF2 level or activity in a test sample from the subject suspected of having skin cancer and comparing the level to ATF2 levels or activities in samples from subjects of known skin cancer stage or type, wherein an ATF2 level or activity about equal to the level or activity in the known skin cancer sample characterizes the skin cancer stage or type of the subject.

22-53. (canceled)
